(19) United States
(12) Patent Application Publication
(10) Pub. No.: US 2021/0396729 A1
(43) Pub. Date: Dec. 23, 2021

(54) SMALL AREA REAL-TIME AIR POLLUTION ASSESSMENT SYSTEM AND METHOD

(71) Applicant: DATAA DEVELOPMENT CO., LTD., Taipei City (TW)

(72) Inventors: Kuang-Fu Cheng, Taipei City (TW); Ya-Hui Yang, Taipei City (TW); Chih-Shen Li, Taipei City (TW); Gui-Han Liu, Taipei City (TW); Deng-Yang Wu, Taipei City (TW)

(21) Appl. No.: 16/908,760

(22) Filed: Jun. 23, 2020

(51) Int. Cl. G01N 33/00; A61B 5/0205; G16H 70/60; G16H 50/70; G16H 10/20; G16H 50/50; G16H 50/30; G16H 10/60

(52) U.S. Cl. CPC G01N 33/0075; A61B 5/0205; G16H 70/60; G16H 50/70; A61B 5/021; G16H 50/50; G16H 50/30; G16H 10/60; A61B 2560/0242; G16H 10/20

(57) ABSTRACT

A small area real-time air pollution assessment system includes a databank, a model generation module, an input module and an analysis module. In a small area real-time air pollution assessment method, the model generation module generates a model by analyzing historical tested persons' historical body characteristics data and historical air data in a plurality of monitored areas, which storing in a databank; inputs from an input module to the model a plurality of current tested persons' body characteristics data in a to-be-monitored area to generate air data corresponding to the current tested persons; selects a specified value of each of those air data and converts the specified values into air quality index values; selects a specific value of those air quality index values; and compares the specific value with an air quality health assessment table to generate assessment results. Thus, relatively accurate small area air pollution assessment results can obtainable.

### FIELD OF THE INVENTION

The present invention relates to a system and a method for air pollution assessment, and more particularly, to a system and a method for small area real-time air pollution assessment.

### BACKGROUND OF THE INVENTION

Presently, the air quality monitoring relies on the air monitoring stations set up at different places. Air data collected by the air monitoring stations are transmitted to a central office and are computed to obtain large-area air pollution assessment results.

However, the air monitoring stations are not evenly distributed in different areas. For example, in Taiwan, Hsinchu county and Hsinchu city have fewer air monitoring stations than Taipei city, and Tainan city has fewer air monitoring stations than Kaohsiung city. Due to the uneven distribution of domestic air monitoring stations, the government can only provide the generate public with large area air pollution assessment results. As to the areas having fewer air monitoring stations, only speculated instead of accurate assessment results can be obtained for them. Besides, the existing monitoring station distribution mode can only be used to assess the air pollution in large areas, such as north Taiwan and south Taiwan, but not in small areas, such as many suburbs, towns, cities, districts and villages in Taiwan. These small areas can only obtain speculated air pollution assessment results instead of relatively accurate assessment results for each of them.

It is therefore tried by the inventor to develop a small area real-time air pollution assessment system and method, with which relatively accurate small area air pollution assessment results can be obtained.

### SUMMARY OF THE INVENTION

A primary object of the present invention is to provide a small area real-time air pollution assessment method, with which relatively accurate small area air pollution assessment results can be obtained.

To achieve the above and other objects, the present invention provides a small area real-time air pollution assessment system, which includes a databank storing a plurality of historical body characteristics data of a plurality of historical tested persons and a plurality of historical air data, which are collected in a plurality of monitored areas, and an air quality-health impacts assessment table; a model generation module being connected to the databank and analyzing those historical body characteristics data and those historical air data to generate a model; an input module for providing a plurality of body characteristics data of a plurality of current tested persons in a to-be-monitored area; and an analysis module being connected to the databank, the model generation module and the input module for inputting the body characteristics data of the current tested persons to the model to generate a plurality of air data that are corresponding to the current tested persons, selecting a specified value in those air data for converting into a plurality of air quality index values, selecting a specific value in those air quality index values; and comparing the specific value with the air quality-health impacts assessment table to generate assessment results.

To achieve the above and other objects, the present invention further provides a small area real-time air pollution assessment method, which includes the steps of using a model generation module to generate a model by analyzing a plurality of historical body characteristics data of a plurality of historical tested persons and a plurality of historical air data, which are collected in a plurality of monitored areas and are stored in a databank; using an analysis module to input from an input module a plurality of body characteristics data of a plurality of current tested persons in a to-be-monitored area to the model for generating a plurality of air data that are corresponding to the current tested persons; using the analysis module to select a specified value in each of those air data and convert the specified values into a plurality of air quality index values; using the analysis module to select a specific value of those air quality index values; and using the analysis module to compare the specific value with the air quality-health impacts assessment table to generate assessment results.

In the method of present invention, since the data analyzed are the tested persons' body characteristics data collected from a small area, such as a suburb, a town, a city, a district or a village, that has not or fewer air monitoring stations, relatively accurate air pollution assessment results can be obtained for the small area.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of a small area real-time air pollution assessment system according to an embodiment of the present invention;

FIG. 2 is a flow chart showing the steps included in a small area real-time air pollution assessment method according to an embodiment of the present invention;

FIG. 3 is a conceptual view showing some examples of air monitored areas referred to in the small area real-time air pollution assessment system and method according to the present invention;

FIG. 4 shows an implementation example of the small area real-time air pollution assessment method according to the present invention;

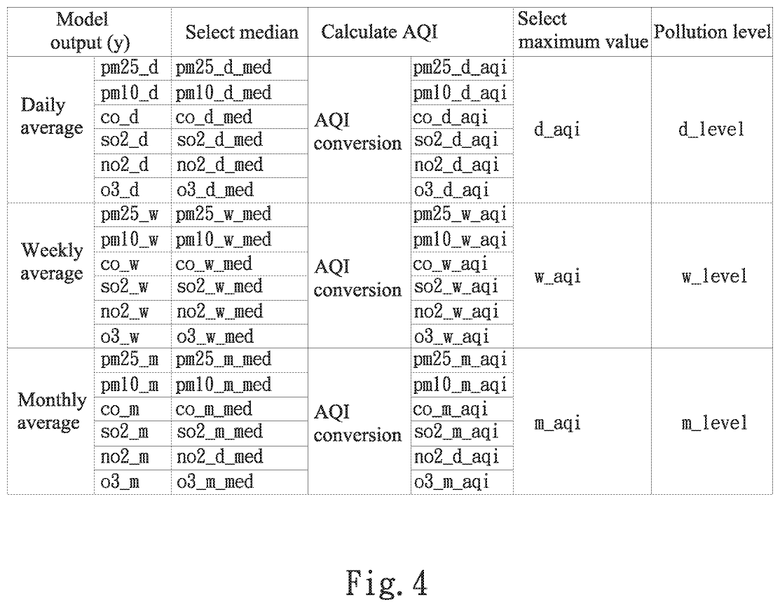

FIG. 5 shows the air quality index values (AQIs) conversion according to the small area real-time air pollution assessment method of the present invention; and

FIG. 6 is an AQI Health Impacts table obtained using the small area real-time air pollution assessment system and method according to the present invention.

### DETAILED DESCRIPTION OF THE PREFERRED EMBODIMENTS

The present invention will now be described with some preferred embodiments thereof and by referring to the accompanying drawings.

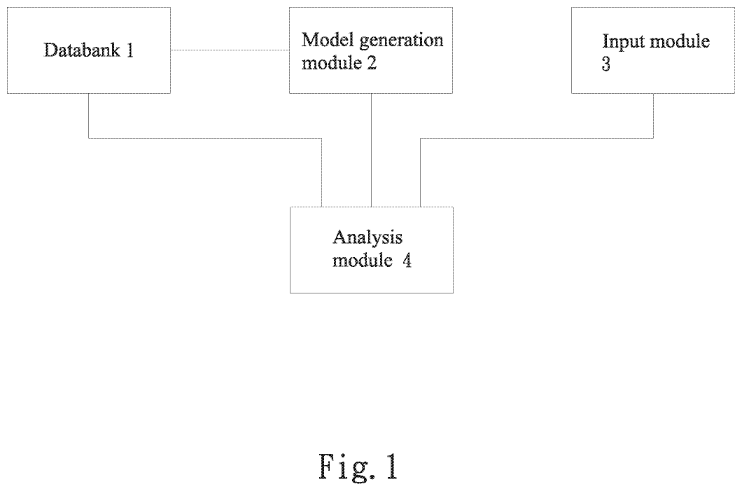

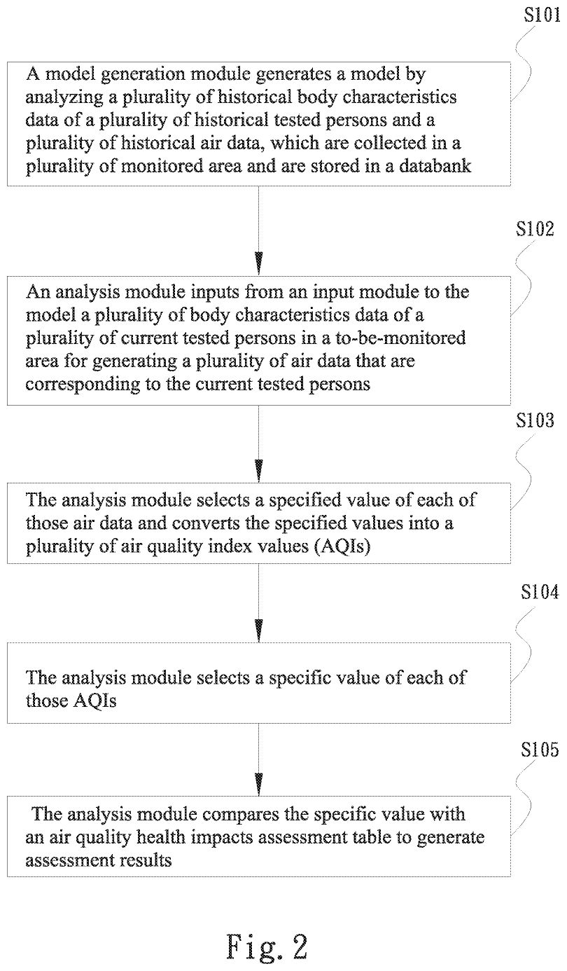

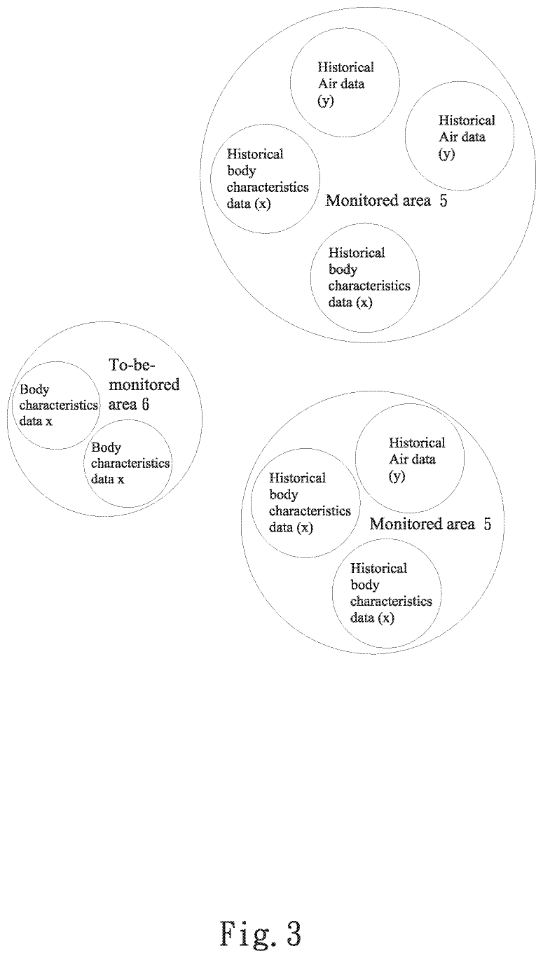

Please refer to FIG. 1 that is a block diagram of a small area real-time air pollution assessment system according to an embodiment of the present invention, and to FIG. 2 that is a flow chart showing the steps included in a small area real-time air pollution assessment method according to an embodiment of the present invention, and to FIG. 3 that is a conceptual view showing some examples of air monitored areas referred to in the small area real-time air pollution assessment system and method according to the present invention. As shown, the small area real-time air pollution assessment system of the present invention includes at least a databank 1, a model generation module 2, an input module 3 and an analysis module 4.

The analysis module 4 is connected to the databank 1, the model generation module 2 and the input module 3; and the model generation module 2 is also connected to the databank 1. The databank 1 as well as the model generation module 2, the input module 3 and the analysis module 4 can be realized through, for example, electronic circuits with special functions or hardware devices having special firmware that are connected to one another. In the case of being realized by software, the model generation module 2, the input module 3 and the analysis module 4 can be non-transitory computer program products with program codes. Since computer program products can be loaded into a microprocessor or a microcontroller for the same to execute specific operations, the computer program products can also considered as special functional modules of the microprocessor or the microcontroller. In an embodiment of the present invention, the model generation module 2, the input module 3 and the analysis module 4 can be programs independent of one another, or can be subprograms of one program. The program codes of the model generation module 2, the input module 3 and the analysis module 4 can be created using various program languages. According to an embodiment, the databank 1 as well as the model generation module 2, the input module 3 and the analysis module 4 can be located in the same or in different devices. For instance, the databank 1 as well as the model generation module 2, the input module 3 and the analysis module 4 can be located in the same server or computer and connected to one another. Alternatively, the databank 1 can be a non-transitory computer-readable medium, such as an optical disk, a hard disk drive or a flash drive, or can be located in a cloud server. Then, data transmission among the databank 1 and the model generation module 2, the input module 3 and the analysis module 4 is performed through wired or wireless connection among them.

The databank 1 stores a plurality of historical body characteristics data of a plurality of historical tested persons, which are independent variables x, and a plurality of historical air data, which are dependent variables y, collected in a plurality of monitored areas 5 of an air monitoring station, and an air quality-health impacts assessment table. Each of the historical body characteristics data includes a plurality of body characteristics items, which include 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight. The 6MWD and the heart rate are measured for example using a wearable device; the DBP and the SBP are measured for example using a blood pressure meter; the oxygen saturation is measured for example using a pulse oximeter; and the FEV₁, the FVC and the PEFR are measured for example using a spirometer. These measuring devices can communicate with the databank 1 through wired or wireless connection, and transmit the measured data to the databank 1.

Each of the historical air data includes a plurality of daily, weekly and monthly air substance items. The air substance items include fine particulate matters (PM_{2.5}), particulate matters (PM₁₀), carbon monoxide (CO), sulfur dioxide (SO₂), nitrogen dioxide (NO₂) and ozone (O₃), such as the daily average of the fine particulate matters (PM_{2.5}), the particulate matters (PM₁₀), the carbon monoxide (CO), the sulfur dioxide (SO₂), the nitrogen dioxide (NO₂) and the ozone (O₃), the weekly average of the fine particulate matters (PM_{2.5}), the particulate matters (PM₁₀), the carbon monoxide (CO), the sulfur dioxide (SO₂), the nitrogen dioxide (NO₂) and the ozone (O₃), and the monthly average of the fine particulate matters (PM_{2.5}), the particulate matters (PM₁₀), the carbon monoxide (CO), the sulfur dioxide (SO₂), the nitrogen dioxide (NO₂) and the ozone (O₃). The air substance items are open data collected by air monitoring stations established by the government at different areas. The unit of the fine particulate matter (PM_{2.5}) is microgram per cubic meter (μg/m³), the unit of the particulate matter (PM₁₀) is μg/m³, the unit of the carbon monoxide (CO) is parts-per-million (ppm), the unit of the sulfur dioxide (SO₂) is parts-per-billion (ppb), the unit of the nitrogen dioxide (NO₂) is ppb, and the unit of the ozone (O₃) is also ppb.

$${\hat{y}}_{i} = {b}_{o} + {b}_{1} {x}_{i}$$

$${b}_{0} = \overline{y} - {b}_{1} \overline{x}$$

$${b}_{1} = \frac{{\sum }_{i = 1}^{n} ( {y}_{i} - \overline{y} ) {x}_{i}}{{\sum }_{i = 1}^{n} ( {x}_{i} - \overline{x} ) {x}_{i}} = \frac{{\sum }_{i = 1}^{n} ( {y}_{i} {x}_{i} ) - n \overline{y} \overline{x}}{{\sum }_{i = 1}^{n} ( {x}_{i} {x}_{i} ) - n \overline{x} \overline{x}} = \frac{{\sum }_{i = 1}^{n} ( {y}_{i} - \overline{y} ) ( {x}_{i} - \overline{x} )}{{\sum }_{i = 1}^{n} {( {x}_{i} - \overline{x} )}^{2}}$$

The model generation module 2 analyzes those historical body characteristics data and those historical air data to generate a model, i.e. parameters b₀, b₁ are generated by calculating the independent variables x and the dependent variables y. In the illustrated embodiment of the present invention, the model generation module 2 uses regression analysis to analyze those historical body characteristics data and those historical air data to generate the model. Herein, the model is a regression model. However, it is understood that, in other operable embodiments, the model generation module 2 can use other mathematical analyses to generate other mathematical models.

The input module 3 provides a plurality of body characteristics data, which are independent variables x, of a plurality of current tested persons within a to-be-monitored area 6 that has not or fewer monitoring stations (see FIG. 2, S101). Each of the body characteristics data includes a plurality of body characteristics items, which include 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight. Since these body characteristics items are measured in the same way as the above-mentioned historical body characteristics items, this part is not repeatedly described herein.

There is not any data collection time interval particularly set for the current tested persons. The current tested persons' body characteristics data can be input via the input module 3 at any time for real-time small area air pollution assessment.

The analysis module 4 inputs the current tested persons' body characteristics data to the model for generating a plurality of air data, which are dependent variables y and corresponding to those current tested persons (see FIG. 2, S102). Each of the air data corresponding to the current tested persons are shown in a plurality of intervals of time. The air data include a plurality of daily, weekly and monthly air substance items. The air substance items include fine particulate matters (PM_{2.5}), particulate matters (PM₁₀), carbon monoxide (CO), sulfur dioxide (SO₂), nitrogen dioxide (NO₂) and ozone (O₃), such as the daily average of the fine particulate matters (PM_{2.5}), the particulate matters (PM₁₀), the carbon monoxide (CO), the sulfur dioxide (SO₂), the nitrogen dioxide (NO₂) and the ozone (O₃), the weekly average of the fine particulate matters (PM_{2.5}), the particulate matters (PM₁₀), the carbon monoxide (CO), the sulfur dioxide (SO₂), the nitrogen dioxide (NO₂) and the ozone (O₃), and the monthly average of the fine particulate matters (PM_{2.5}), the particulate matters (PM₁₀), the carbon monoxide (CO), the sulfur dioxide (SO₂), the nitrogen dioxide (NO₂) and the ozone (O₃). Since the units of these air substance items are the same as those of the air substance items of the historical air quality data, they are not repeatedly described herein.

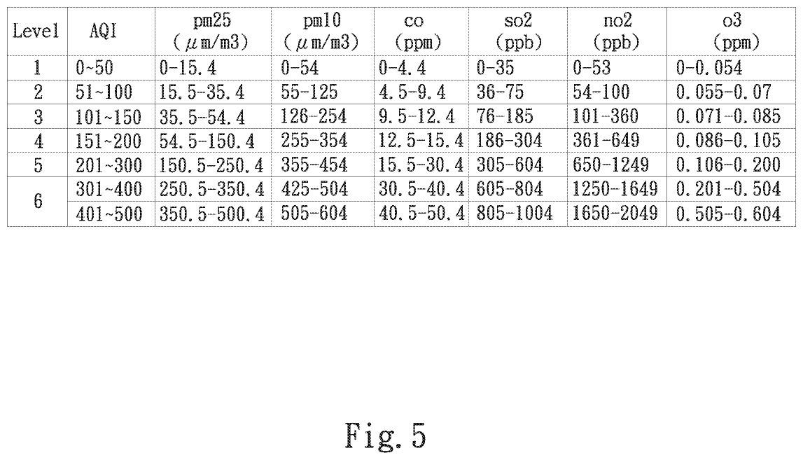

Further, the analysis module 4 selects a specified value in each of those air data and converts the specified values into a plurality of air quality index values. In the illustrated embodiment, the air quality index values are shown as the air quality index (AQI). In other embodiments, the air quality index value can be shown as air quality health index (AQHI), air pollution index (API), comprehensive air-quality index (CAI) or common air quality index (CAQI) (see FIG. 2, S103 and FIG. 5). The number of specific values of the air quality index values is corresponding to the number of the intervals of time; and the number of assessment results is also corresponding to the number of the intervals of time. According to the degree of human health impacts of the values of the day's fine particulate matters (PM_{2.5}), particulate matters (PM₁₀), carbon monoxide (CO), sulfur dioxide (SO₂), nitrogen dioxide (NO₂) and ozone (O₃) shown in the monitored data, sub-indices for different pollutants in the day are obtained. In the illustrated embodiment, the analysis module 4 selects a median of each of those sub-indices in the air data and converts the medians into the plurality of air quality index values. However, it is understood the above embodiment is non-restrictive. In other operable embodiments, the analysis module 4 can select an average of each of those sub-indices in the air data and covert the averages into the plurality of air quality index values.

In addition, the analysis module 4 selects a specific value of those air quality index values (see FIG. 2, S104). In the illustrated embodiment, the analysis module 4 is shown to select the maximum value of the plurality of air quality index values and use it as the day's air quality index values. However, it is understood the above embodiment is non-restrictive. In other operable embodiments, the analysis module 4 can select other value in those air quality index values to serve as the day's air quality index values.

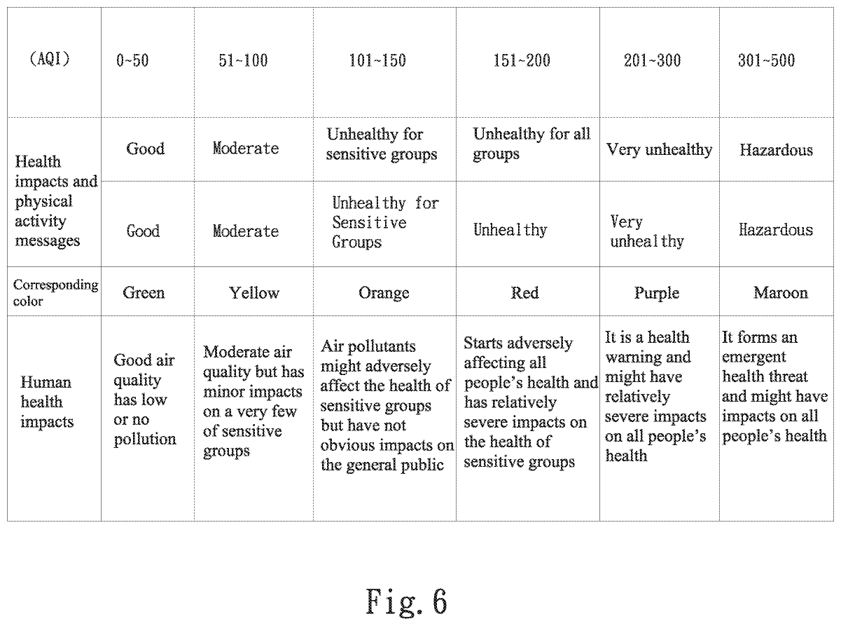

Then, the analysis module 4 compares the specific value of those air quality index values with the air quality-health impacts assessment table to generate assessment results (see FIG. 2, S105). The assessment results are the results of air pollution assessment of the to-be-monitored area 6. The air quality-health impacts assessment table can be, for example, the AQI and Health Impacts Table (see FIG. 6) published by Taiwan Environmental Protection Administration. However, it is understood the present invention is not limited thereto. In other operable embodiments, the air quality-health impacts assessment table can be an air quality health table published by any other governmental agency, institute or research organization.

In the small area real-time air pollution assessment system and method of the present invention, the historical body characteristics data and the historical air data of the monitored areas having air monitoring stations are analyzed to generate the regression model, and the body characteristics data of the current tested persons in the to-be-monitored areas are input to the regression model to generate the air data of the to-be-monitored areas; and the air data of the to-be-monitored areas are converted into the plurality of air quality index values; and lastly, the plurality of air quality index values are compared with the air quality-health impacts assessment table to determine an air condition level of the to-be-monitored areas. In the small areas, such as suburban areas, towns, cities, districts and villages, which have not or fewer air monitoring stations, accurate air pollution assessment can be achieved by analyzing the body characteristics data of tested persons in the to-be-monitored areas.

Compared to the conventional way of using the air data collected in areas having a relatively large number of monitoring stations to assess the air data of small areas having not or fewer monitoring stations, the present invention can provide more accurate assessment results of the air pollution in small areas.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A small area real-time air pollution assessment system, comprising:
a databank storing a plurality of historical body characteristics data of a plurality of historical tested persons and a plurality of historical air data, which are collected in a plurality of monitored areas, and an air quality-health impacts assessment table;
a model generation module being connected to the databank and analyzing those historical body characteristics data and those historical air data to generate a model;
an input module for providing a plurality of body characteristics data of a plurality of current tested persons in a to-be-monitored area; and
an analysis module being connected to the databank, the model generation module and the input module for inputting the body characteristics data of the current tested persons to the model to generate a plurality of air data that are corresponding to the current tested persons, selecting a specified value in each of those air data for converting into a plurality of air quality index values, selecting a specific value in those air quality index values for comparing with the air quality-health impacts assessment table to generate assessment results.

2. The small area real-time air pollution assessment system as claimed in claim 1, wherein the air data corresponding to the current tested persons are divided according to a plurality of intervals of time, the number of the specific values selected from those air quality index values is corresponding to the number of the intervals of time, and the number of the assessment results is also corresponding to the number of the intervals of time.

3. The small area real-time air pollution assessment system as claimed in claim 2, wherein the intervals of time include a day, a week and a month.

4. The small area real-time air pollution assessment system as claimed in claim 1, wherein the historical body characteristics data include a plurality of body characteristics items; and the body characteristics items including 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight.

5. The small area real-time air pollution assessment system as claimed in claim 1, wherein the historical air data includes a plurality of daily, weekly and monthly air substance items; and the air substance items including fine particulate matters (PM_{2.5}), particulate matters (PM₁₀), carbon monoxide (CO), sulfur dioxide (SO₂), nitrogen dioxide (NO₂) and ozone (O₃).

6. The small area real-time air pollution assessment system as claimed in claim 1, wherein the body characteristics data of the current tested persons in a to-be-monitored area include a plurality of body characteristics items; and the body characteristics items including 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight.

7. The small area real-time air pollution assessment system as claimed in claim 1, wherein the air data corresponding to the current tested persons include a plurality of daily, weekly and monthly air substance items; and the air substance items including fine particulate matters (PM_{2.5}), particulate matters (PM₁₀), carbon monoxide (CO), sulfur dioxide (SO₂), nitrogen dioxide (NO₂) and ozone (O₃).

8. The small area real-time air pollution assessment system as claimed in claim 1, wherein the specified value can be any one of an average and a median of each of those air data corresponding to the current tested persons.

9. The small area real-time air pollution assessment system as claimed in claim 1, wherein the specific value is a maximum value of those air quality index values.

10. The small area real-time air pollution assessment system as claimed in claim 1, wherein the model generation module uses regression analysis to analyze those historical body characteristics data and those historical air data to generate the model; and the model being a regression model.

11. A small area real-time air pollution assessment method, comprising the following steps of:
using a model generation module to generate a model by analyzing a plurality of historical body characteristics data of a plurality of historical tested persons and a plurality of historical air data, which are collected in a plurality of monitored area and are stored in a databank;
using an analysis module to input from an input module to the model a plurality of body characteristics data of a plurality of current tested persons in a to-be-monitored area for generating a plurality of air data that are corresponding to the current tested persons;
using the analysis module to select a specified value of each of those air data and convert the specified values into a plurality of air quality index values;
using the analysis module to select a specific value of each of those air quality index values; and
using the analysis module to compare the specific value with the air quality-health impacts assessment table to generate assessment results.

12. The small area real-time air pollution assessment method as claimed in claim 11, wherein the air data corresponding to the current tested persons are divided according to a plurality of intervals of time, the number of the specific values selected from those air quality index values is corresponding to the number of the intervals of time, and the number of the assessment results is also corresponding to the number of the intervals of time.

13. The small area real-time air pollution assessment method as claimed in claim 12, wherein the intervals of time include a day, a week and a month.

14. The small area real-time air pollution assessment method as claimed in claim 11, wherein the historical body characteristics data include a plurality of body characteristics items; and the body characteristics items including 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight.

15. The small area real-time air pollution assessment method as claimed in claim 11, wherein the historical air data includes a plurality of daily, weekly and monthly air substance items; and the air substance items including fine particulate matters (PM_{2.5}), particulate matters (PM₁₀), carbon monoxide (CO), sulfur dioxide (SO₂), nitrogen dioxide (NO₂) and ozone (O₃).

16. The small area real-time air pollution assessment method as claimed in claim 11, wherein the body characteristics data of the current tested persons in a to-be-monitored area include a plurality of body characteristics items; and the body characteristics items including 6-minute walking distance (6MWD), heart rate, diastolic blood pressure (DBP), systolic blood pressure (SBP), oxygen saturation, forced expiratory volume in one second (FEV₁), forced vital capacity (FVC), peak expiratory flow rate (PEFR), sex, age, height and weight.

17. The small area real-time air pollution assessment method as claimed in claim 11, wherein the air data corresponding to the current tested persons include a plurality of daily, weekly and monthly air substance items; and the air substance items including fine particulate matters (PM_{2.5}), particulate matters (PM₁₀), carbon monoxide (CO), sulfur dioxide (SO₂), nitrogen dioxide (NO₂) and ozone (O₃).

18. The small area real-time air pollution assessment method as claimed in claim 11, wherein the specified value can be any one of an average and a median of each of those air data corresponding to the current tested persons.

19. The small area real-time air pollution assessment method as claimed in claim 11, wherein the specific value is a maximum value of those air quality index values.

20. The small area real-time air pollution assessment method as claimed in claim 11, wherein the model generation module uses regression analysis to analyze those historical body characteristics data and those historical air data to generate the model; and the model being a regression model.
